(19) United States
(12) Patent Application Publication
(10) Pub. No.: US 2013/0211529 A1
(43) Pub. Date: Aug. 15, 2013

(54) JOINT AUGMENTATION IMPLANT AND APPLICATIONS THEREOF

(71) Applicant: SOFTJOINT CORPORATION, (US)

(72) Inventors: John T. Frauens, Honolulu, HI (US); Phillip M. Leopold, North Barrington, IL (US); Tanner Hargens, Charlotte, NC (US)

(73) Assignee: SOFTJOINT CORPORATION, Honolulu, HI (US)

(21) Appl. No.: 13/760,161

(22) Filed: Feb. 6, 2013

Related U.S. Application Data
(60) Provisional application No. 61/597,402, filed on Feb. 10, 2012.

(51) Int. Cl. A61F 2/30

(52) U.S. Cl. CPC A61F 2/30
USPC 623/18.11

(57) ABSTRACT

The disclosure relates to a joint augmentation implant in the form of an elastomeric polymer cap for the ball portion of a ball and socket joint such as a hip or shoulder. The cap is adapted to swell in the presence of synovial fluid and to supplement or replace native cartilage. The disclosure also relates to methods of use of the joint augmentation implant.

### BACKGROUND

Osteoarthritis is the leading cause for joint replacement surgery worldwide. Although the bone may eventually be involved, osteoarthritis is primarily a disease of cartilage. Bones have sensory nerves just like skin. These nerves exist on the surfaces of the bone both on the femoral head and acetabulum. Normally the bone surfaces along with their sensory nerves are covered by articular cartilage or hyaline cartilage. Hyaline cartilage is unique in that not only does it not have a blood supply, it also does not possess a nerve supply, i.e., it is aneural. Therefore, as long as there is cartilage interposed between the joint surfaces, since there are no nerves, there is no pain. The pain of osteoarthritis is generated once the cartilage has eroded away, and there is resulting bone on bone contact or nerve on nerve contact.

There are three basic classifications of joints of the human body: synarthroidal, amphiarthroidal, and diarthroidal. Synarthroidal joints provide immovable articulations; amphiarthroidal joints provide mixed articulations; and diarthroidal joints provide movable articulations. Healthy fibro cartilage and hyaline cartilage within the joint provide a weight-bearing function and allow painless articulation of amphiarthroidal and diarthroidal joints.

Primary osteoarthritis is a debilitating disease that affects amphiarthroidal and diarthroidal joints. The changes that occur with primary osteoarthritis involve altered biomechanical, biochemical, histological and metabolic characteristics of the cartilage, synovial fluid and bone. Initially, these changes affect the articular cartilage and eventually affect the surrounding perichondral tissues in a cascade of events.

Articular cartilage, also called hyaline cartilage, is made of a multiphasic material with two major phases: a fluid phase composed of water (68%-85%) and electrolytes, and a solid phase composed of collagen fibrils (primarily type II collagen, 10%-20%), proteoglycans and other glycoproteins (5-10%), and chrondrocytes (cartilaginous cells). 30% of all cartilage water resides in this interstitial fluid, and this amount does not vary with age. However, there is a significant increase of total amount of water in degenerating cartilages. This multiphasic system allows fluid flowing from the tissue to the solution surrounding the tissue, and vice versa, through the pores of the collage-proteoglycan solid matrix. As the fluid passes to the pores, the force exerted on the walls of the pores causes more compaction. Thus, it becomes more and more difficult to squeeze fluid from the tissue with prolonged compression. This non-linear flow-induced compression effect is very important in the physiology of cartilage not just because it determines cartilage compressive behaviors, but also because it provides the mechanism for energy dissipation.

There are many theories concerning how articular cartilage functions as a weight bearing surface, which include hydrodynamic, boundary, elastohydrodynamic and squeeze film lubrication. However, it is known that the viscoelastic properties contribute to the multiple functions of articular cartilage, including its weight bearing function. The viscoelastic properties of cartilage are due to an intricate tight meshwork of interlacing collagen fibers that physically ensnare the large macromolecules of proteoglycan.

To date, treatment of osteoarthritis of the hip joint has been with the use of total joint replacement surgery. This entails resection of the proximal femur (femoral head and neck), reaming of the femoral intramedullary canal and the insertion of one or more modular artificial metal component(s) to replace the diseased cartilage on the resected bone. Similarly the acetabulum is removed by reaming the socket down to bleeding bone and the impacting of an artificial socket into the pelvis. The two components are then joined by suturing the dissected surrounding tissues together, joining the two components into contact with each other. The materials used for these devices are usually an alloy of various metals typically cobalt, chrome and titanium. The bearing surfaces vary from polyethylene on metal, metal on metal, ceramic on ceramic, and various combinations of them all. The operations are extensive dissections with implantation of large quantities of inert material into the human body. Potential complications are extensive and can range anywhere from minor wound complications to death of the patient. Such approaches entail the complete replacement and substitution of the joint with artificial components with their own inherent mechanics of joint function.

This total hip replacement has a relatively long but finite life; the average implant is expected to last 10 to 15 years before it must be revised. To decrease the likelihood of a more arduous and costly revision of the original joint replacement in later years, patients are encouraged to instead use non-surgical treatments when suffering from osteoarthritis at an early age (younger than 65). This practice expects the patient to be far less active or deceased before a revision is required.

Revision procedures average nearly twice the total cost of primary joint replacements due largely to more elderly patients needing additional days in the hospital to recover. This enormous economic burden could be relieved by reducing the number of hip replacement revision procedures and by reducing the numbers of days spent in the hospital recovering from any surgical treatment of hip osteoarthritis.

Similar considerations apply to treatment of the shoulder joint which also relies upon a ball and socket joint which is subject to osteoarthritis. The ball portion is formed by the head of the humerus and a shallow socket is formed by the scapula.

There is a clear clinical need for a bone conserving outpatient method to treat osteoarthritis of the hip and shoulder. Such an option would offer a surgical remedy for younger patients suffering from osteoarthritis while keeping the native bone intact to effectively delay the need for a primary joint replacement. Delaying a primary joint replacement would ultimately reduce the rate of revision procedures and relieve the associated economic burden.

It would be desirable to provide a method and apparatus for treating osteoarthritis that minimizes surgical intervention and human tissue resection and substitution.

### SUMMARY

This disclosure pertains to a joint augmentation implant for mammalian ball and socket joints comprising a hollow polymeric cap formed at least in part from a polymer adapted to swell upon absorption of synovial fluid; wherein the hollow polymeric cap comprises a distal region having the general form of a spherical cap, a generally cylindrical proximal neck region, and an intermediate region comprising a generally spherical segment therebetween; further wherein at least a portion of the hollow polymeric cap within the combined regions having the general form of a spherical cap and the intermediate region having the general form of a spherical segment comprises an exterior articulating region and a bone contacting region.

The disclosure further relates to a method of installing an intra-articular joint augmentation implant as artificial cartilage in a ball and socket joint to be restored, the method comprising forming a hollow polymeric cap formed at least in part from a polymer adapted to swell upon absorption of synovial fluid, said hollow polymeric cap comprising a distal region having the general form of a spherical cap, a generally cylindrical proximal neck region, and an intermediate region comprising a generally spherical segment therebetween, wherein the hollow polymeric cap within the combined regions having the general form of a spherical cap and the intermediate region comprises an exterior articulating region and a bone contacting region; hydrating the hollow polymeric cap; surgically exposing the head of the ball of the ball and socket joint through an access wound; installing the hollow polymeric cap over the head of the ball of the ball and socket joint; repositioning the capped head of the ball of the ball and socket joint within the socket of the ball and socket joint with the hollow polymeric cap interposed between the ball of the ball and socket joint and the socket of the ball and socket joint; and surgically closing the access wound.

Implantation of the device requires no bone resection. Since there is no bone resection, any of the established conservative surgical approaches can be utilized without risk of wound extension or surgical complications. With conservative surgical access and bone conservation, the invention offers an alternative outpatient treatment option for younger patients suffering from osteoarthritis while conserving bone for future joint replacement procedures, if ever deemed necessary. The device does not require promotion of soft tissue ingrowth or regrowth as is encouraged with current cartilage repair and regeneration research.

Relative to existing joint replacement procedures, the device of the disclosure can be expected to dramatically reduce average surgery time and average time and charges associated with post-operative care. The total procedural charges for the invention, including hospital fees, will be greatly reduced relative to conventional joint replacement procedures. Minimal instrumentation will be required and the implantation procedure is simple enough that fellowship training will not be necessary, which is critical in emerging markets where there is a shortage of fellowship trained surgeons.

### BRIEF DESCRIPTION OF DRAWINGS

FIG. 1 is a cross-sectional view illustrating a normal hip joint depicting the femoral head (hip bone), the acetabulum (socket), and the joint capsule.

FIG. 2 is a cross-sectional view of an arthritic hip joint illustrating the loss of cartilage surface from both the femoral head and acetabulum.

FIG. 3 is a cross-sectional view showing the femoral head of an arthritic hip that is surgically dislocated from the hip socket and joint capsule for installation of the joint augmentation implant.

FIG. 4 shows an embodiment of the polymeric cap prior to placement over the femoral head.

FIG. 5 shows the polymeric cap positioned over the arthritic femoral head.

FIG. 6 shows the restored arthritic hip with the polymeric cap over the femoral head swollen with synovial fluid to re-establish the joint space and a smooth joint surface.

FIG. 7 shows the bones of a normal shoulder joint.

FIG. 8 shows a simplified perspective view of an embodiment of the disclosure.

FIG. 9 shows a top view of the embodiment of FIG. 8.

FIG. 10 indicates the overall dimensions of a cross-section of an embodiment of the disclosure.

FIG. 11 shows the principle regions of an embodiment of the disclosure.

FIGS. 12A-B illustrate a variety of structural features which may be present and/or combined in an embodiment of the disclosure.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described unless clearly stated to the contrary.

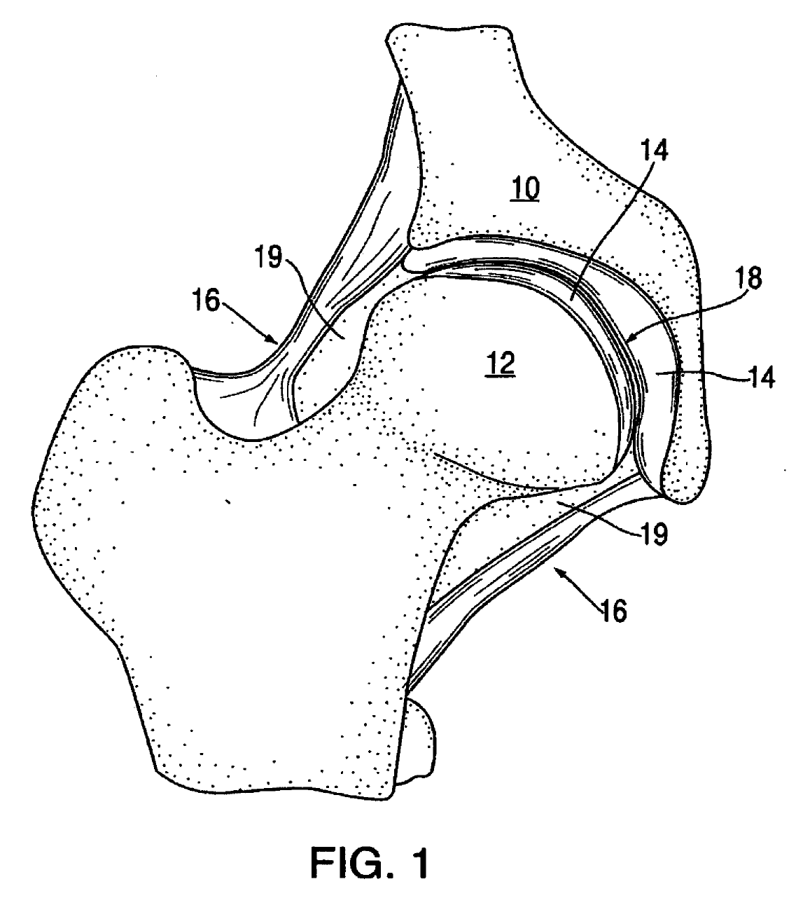

In the following disclosure, attention will be focused on an exemplary human hip joint augmentation implant although the principles disclosed apply to a variety of other joints such as the shoulder joint and other mammalian joints. The terms “joint augmentation implant” and “polymeric cap” may be used interchangeably throughout the disclosure and claims depending on emphasis suggested by context. The hip joint examples are intended to be non-limiting. In FIG. 1, a normal hip joint is shown in cross sectional view with the femoral head 12 of the hip bone positioned in the acetabulum 10 of the hip joint socket with respective layers of articular cartilage 14 forming a smooth joint surface 18 with cartilage-on-cartilage contact. The joint capsule 16 completely encapsulates the hip joint and is filled with synovial fluid 19 that flows to the articular cartilage surfaces 18 of both the femoral head 12 and the acetabulum 10.

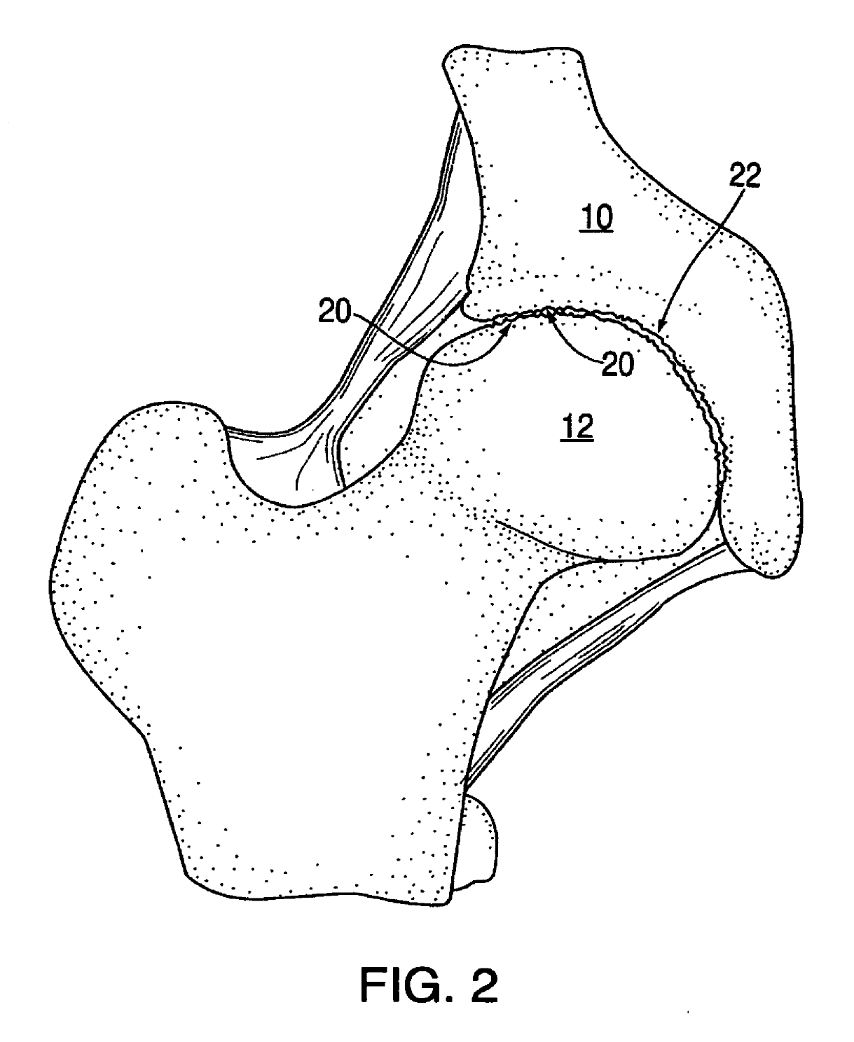

In FIG. 2, an arthritic hip joint is shown in cross sectional view having the articular cartilage of the femoral head 12 and acetabulum 10 worn down on the respective surfaces 20 so that there is raw bone-on-bone contact. This results in contact between the roughened joint surfaces 20 that causes pain. The joint space is greatly reduced due to the lack of cartilage.

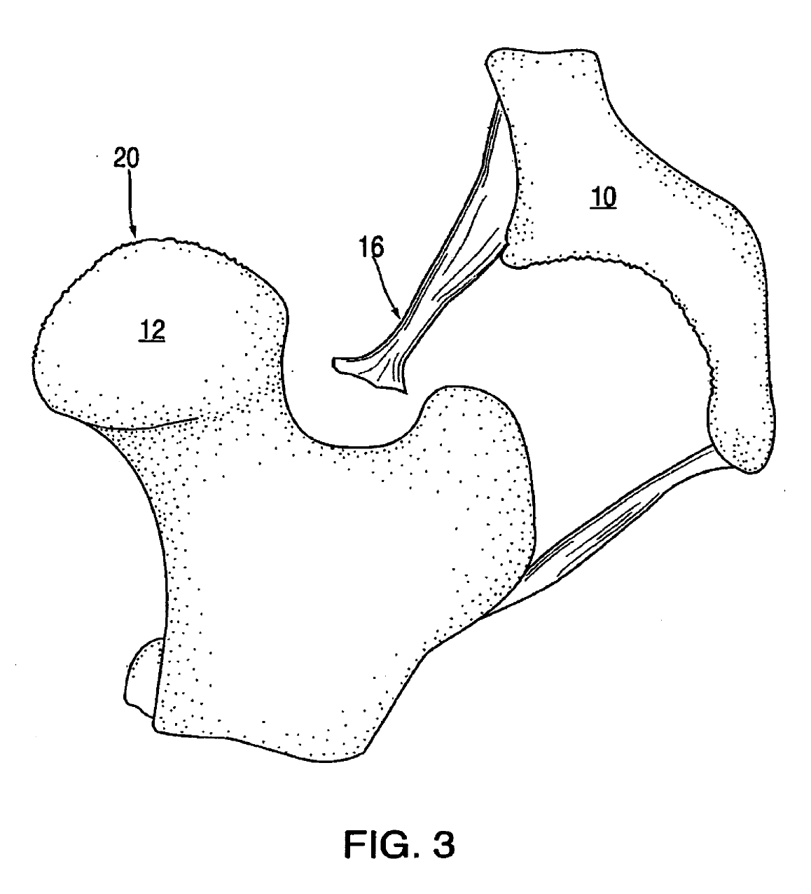

In FIG. 3, a surgical procedure for restoring the arthritic hip in accordance with the present invention temporarily removes (dislocates) the femoral head 12 of the hip bone from the acetabulum 10 outside the joint capsule 16 (by detaching one side) so that the head 12 can be accessed.

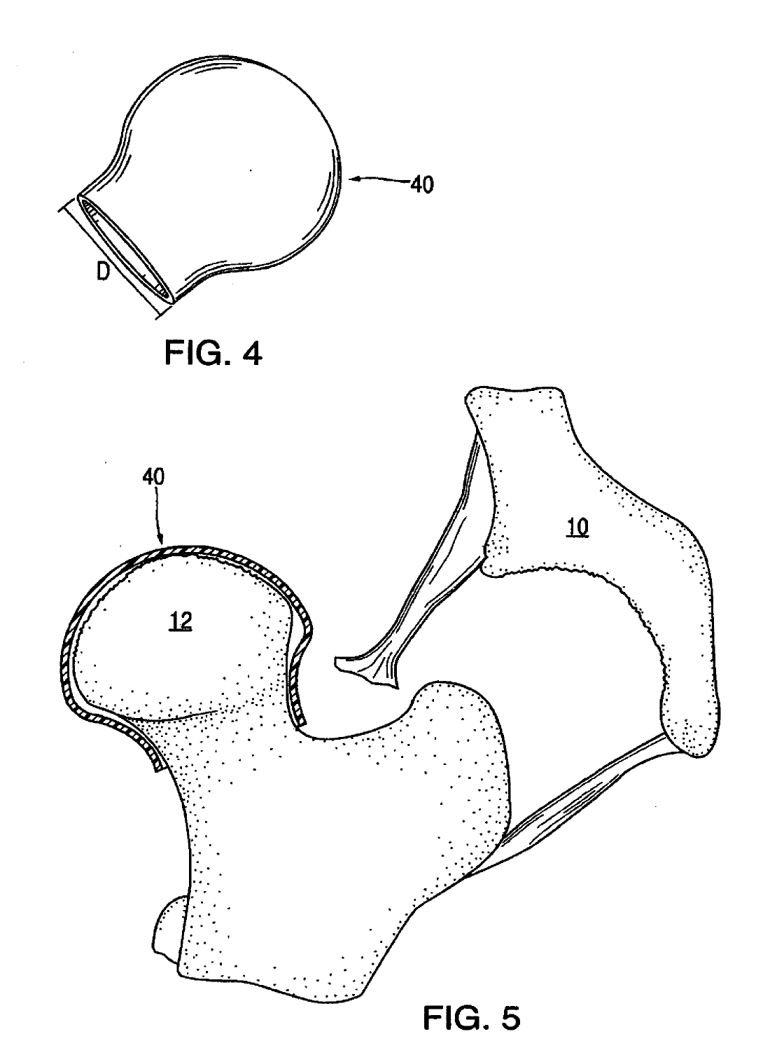

In FIG. 4, a preferred embodiment of an intra-articular joint augmentation implant, in the form of a polymeric cap 40, for placement over the femoral head 12 is illustrated. The intra-articular joint augmentation implant 40 has a balloon shape corresponding to the geometry of the femoral head 12, and the diameter D of its open end is sized to be approximately equal to the diameter of the bone proximate the head and smaller than the balloon portion of the device. Because of the elastic quality of the device material and its shape, the device can be stretched over the femoral head 12 like a condom and secured in place by the open end reverting to its original resting diameter D thereby fitting snugly and securely over the femoral head 12 and neck. It should be understood that the balloon-shaped portion and/or the open end of the device may be minimally stretched, not stretched, or even slightly larger than the femoral head once installed and fully hydrated, although any gaps typically will be minimal.

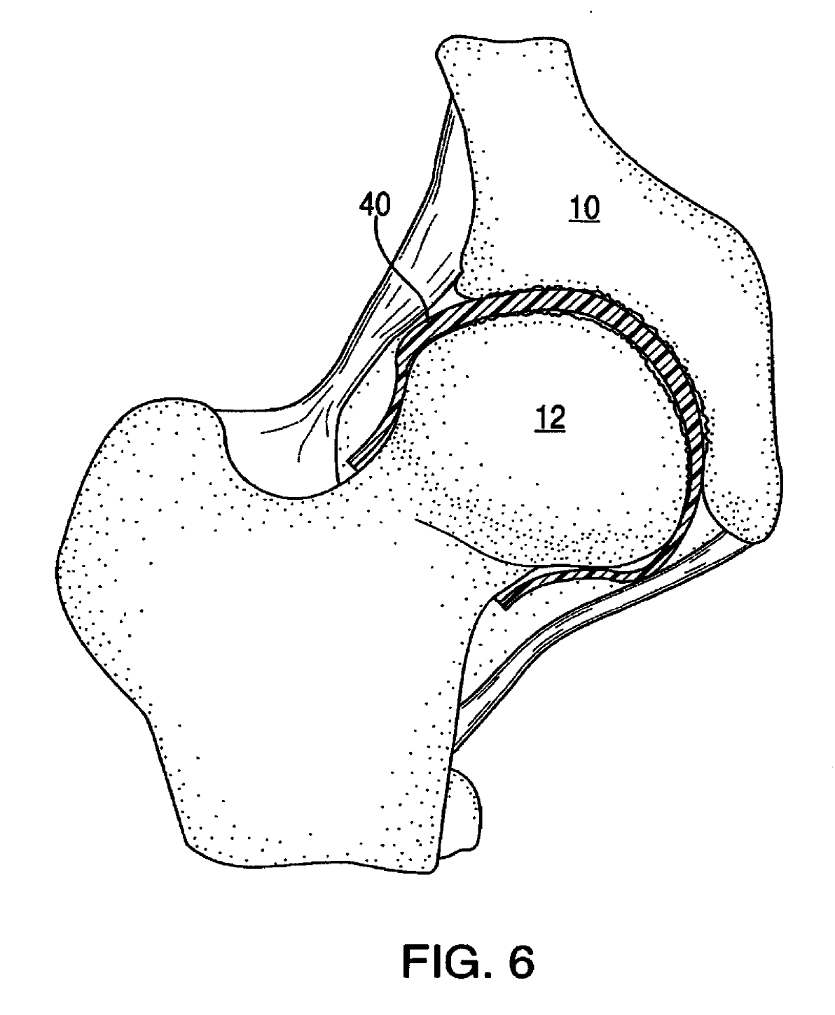

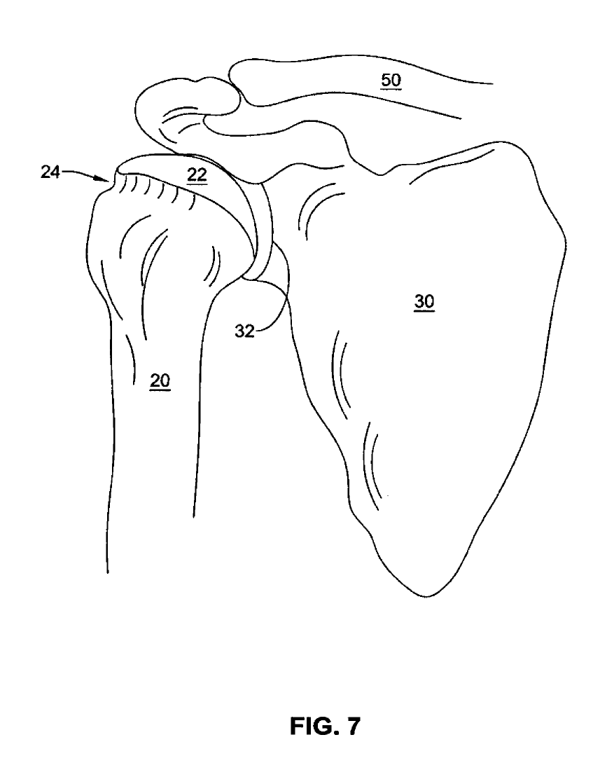

In FIG. 5, the intra-articular joint augmentation implant 40 is shown placed over the femoral head 12 of the hip bone. FIG. 6 shows the restored arthritic hip in which the femoral head 12 has been covered with the joint augmentation implant 40. The joint augmentation implant 40 has become swollen with synovial fluid to interpose a layer of artificial cartilage and re-establish the joint space and a smooth joint surface. FIGS. 5, 6, 10, and 11 do not necessarily include details associated with the inner structure of the hollow polymer cap.

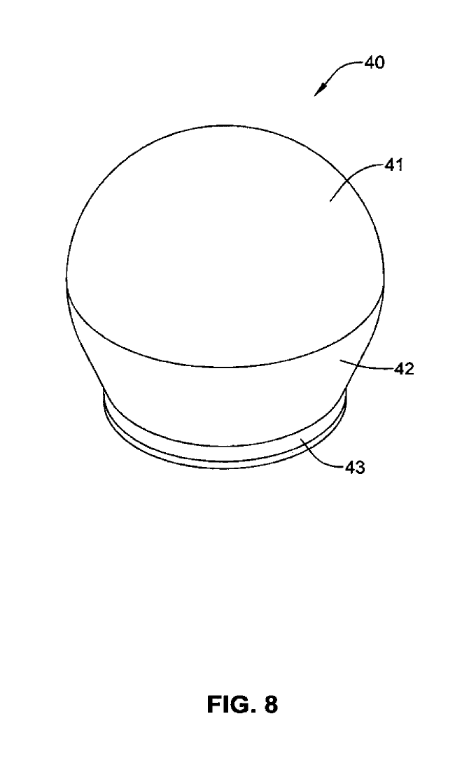

FIG. 7 illustrates the bones of a human shoulder joint. The clavicle 50, humerus 20, humeral head 22, reduced diameter humeral neck portion 24, and scapula 30 with socket, glenoid fossa 32, are depicted without the surrounding soft tissue to more clearly indicate the analogous ball and socket structures also found in shoulder joints. Analogous joints are found in other locations and in other mammals. Such joints, when diseased or damaged, may benefit from the insertion of an intra-articular joint augmentation implant of the disclosure, although the shape and dimensions may need to be altered from the hip augmentation implant described.

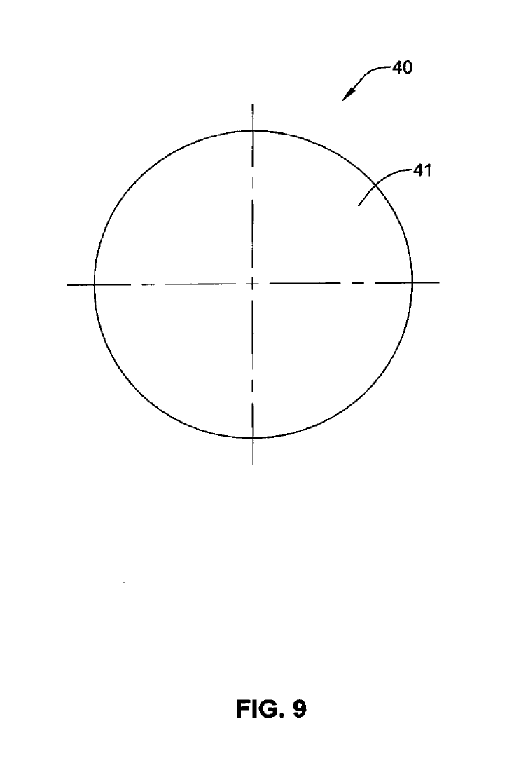

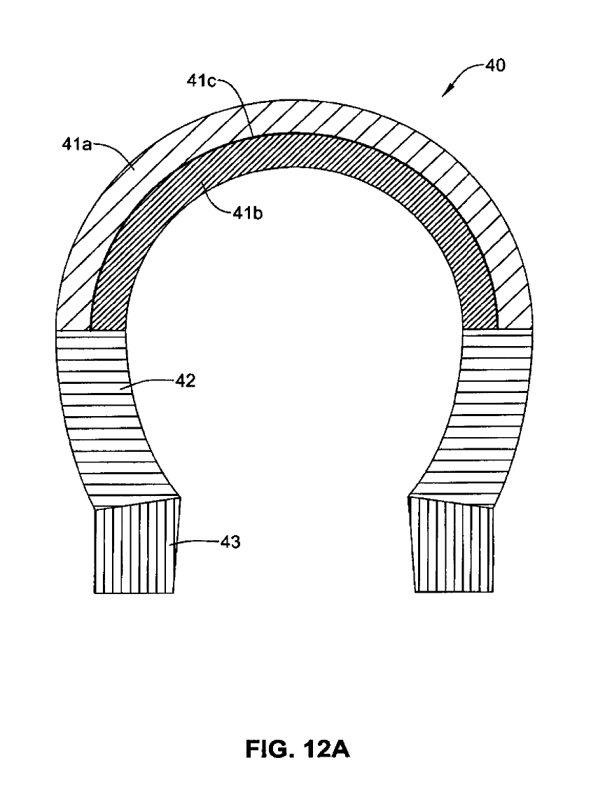

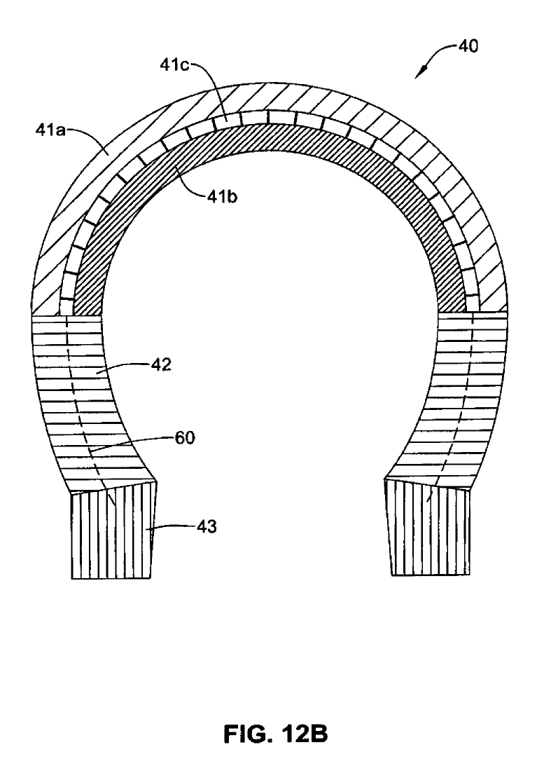

FIGS. 8 and 11 illustrate the three major regions of a polymer cap 40 which forms an intra-articular joint augmentation implant. The polymer cap 40 includes a distal region having the general form of a spherical cap 41 which covers the contact region between the polymer cap, the ball, and the socket of the ball and socket joint to be repaired and generally is responsible for providing the load bearing function. The spherical cap portion 41 of the polymer cap includes further structure to be discussed later with regard to FIG. 12. It will be understood that the distal region 41 of the polymer cap 40 having the general form of a spherical cap may, in some embodiments, comprise more or less than a hemisphere depending upon the configuration and range of motion associated with the ball and socket joint to be repaired.

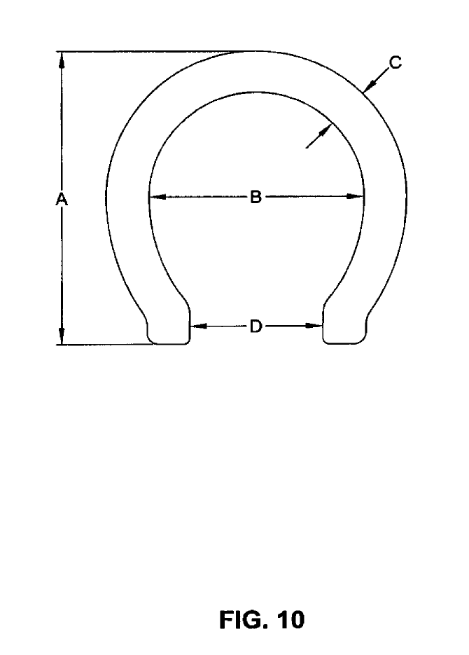

The polymer cap 40 further comprises a generally cylindrical proximal neck region 43 which surrounds the neck of the ball portion of the ball and socket joint to be repaired and serves, in part, to retain the polymer cap 40 in position relative to the ball portion of the joint. The neck portion 43 may include a slight flare or chamfer (which is difficult to see in the figures) to assist in the step of spreading of the opening provided by neck portion 43 as the implant is installed over the ball of the joint. The neck portion 43 of the polymer cap 40 is joined to the spherical cap 41 by an intermediate region comprising a generally spherical segment 42. The intermediate region also will typically contribute to the retention of the polymer cap 40 in position relative to the ball portion of the joint and may participate in the load carrying function. It will be appreciated that the internal radius of the distal spherical cap region 41 and the internal radius of the intermediate spherical segment 42 need not be the same and that the transitions between the distal spherical cap region 41, the intermediate spherical segment 42, and the generally cylindrical proximal neck region 43 may form smooth transitions. The distal spherical cap region 41, intermediate spherical segment 42, and neck portion 43 may be assembled by successive additions of their subcomponents to a common intermediate structure or by first forming the distal spherical cap region 41, intermediate spherical segment 42, and neck portion 43 separately and joining the components by known methods such as, for example, by RF welding. As illustrated in the simplified perspective view of FIG. 8 and the top view of FIG. 9, the polymer cap 40 has radial symmetry about the axis of the cap, as typically will be the case; however in certain embodiments, the polymer cap 40 may be custom shaped to provide an improved fit for atypical or damaged joints.

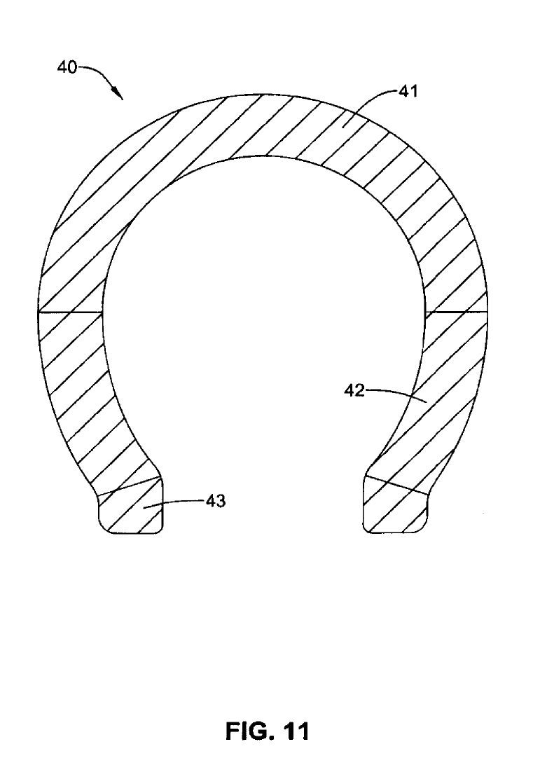

FIG. 10 illustrates typical dimensions associated with an intra-articular joint augmentation implant 40 of the disclosure. It will be understood that that appropriate dimensions for the polymer cap 40 will necessarily vary from recipient to recipient. Dimension “A” of FIG. 10, the overall height of the polymer cap may range from about 10 mm to about 50 mm. Dimensions “B” of FIG. 10, corresponds to the mean radius of the ball portion of the ball and socket joint and thus to the interior dimension of the polymer cap 40. It is contemplated that the internal diameter of the polymer cap 40 will typically be approximately equal to the diameter of the ball portion of the joint, such as a femoral or humeral head. However, in some embodiments, the internal diameter of the polymer cap 40 will have a reduced dimension relative to that of the corresponding bone.

In certain embodiments, the molding fixture about which the polymer cap is formed has a length substantially equal to the length of the intended ball portion, but may include a reduced diameter which is undersized by about 5-25% relative to the diameter of the joint head, with 15% undersized being typical. It has been found that the nominal inner dimensions of the polymer cap remain substantially unchanged as the polymer swells upon exposure to synovial or other liquids. The thickness “C” of the spherical cap and spherical segment may vary in different regions, but typically ranges from 0.50 mm to about 8.00 mm depending on the respective sizes of the joint components and the degree to which some cartilage remains within the intra-articular space such that the overall spherical cap and remaining cartilage substantially replace the original cartilage.

The neck opening, “D”, may be substantially equal to the mean diameter of the ball neck or somewhat undersized and may range from about 0% to about 50% smaller than the neck. In addition, the thickness of the neck region 43 may range between 0.50 mm and 8.0 mm depending upon the material used and the resulting resistance to undesirable expansion which could result in the neck being displaced over the joint head. In any event, the thickness of the neck region 43 should be small enough to avoid mechanical restriction of joint motions.

It should be noted that the intra-articular joint augmentation implants of the disclosure do not require fixation to the bone and, in certain embodiments, a degree of freedom to rotate and/or to exhibit a limited rocking motion may be desirable. In certain embodiments, the intra-articular joint augmentation implants 40 of the disclosure may be fixed to the bone in the neck region or may encourage the development of anchorage through tissue ingrowth. The polymer cap 40 is retained in the proper position, following implantation and initial swelling, by compression between the ball and socket components of the joint, by the elasticity of the polymer cap 40, particularly in the neck region 43, by the increased resistance to deformation as the neck region stretches, and by the tendency of the neck portion to seal the polymer cap to the neck of the bone such that liquid filled spaces, which may be present between the polymer cap and the bone, are responsible for generating a partial vacuum should the polymer cap be displaced from the bone.

Turning to FIGS. 12A and 12B, which illustrate additional structure associated with the spherical cap 41 and, in some embodiments, elements which may extend into the intermediate spherical segment and neck regions, attention is initially drawn to an exterior articulating region 41A and a bone contacting region 41B. The joint augmentation implant is made, at least in part, of a material selected to have a property of absorbing the joint's own synovial fluid to thereby cause the membrane to swell and have a viscoelastic property similar to the body's own articular hyaline cartilage. It will be understood that the exterior articulating region 41A provides the convex outer surface while bone contacting region 41B may be viewed as lining at least a portion of the concave cavity of the joint augmentation implant such that it is generally adjacent to the head of the ball portion of the joint. In some embodiments, there may be an additional layer (not shown) lining the innermost surface of the joint augmentation implant which may serve to lessen contact pressures between the bone contacting region 41B and the head of the joint when the head has been roughened by wear. The additional layer may be considered a component of the bone contacting region 41B. It is not necessarily the case that the bone contacting region 41B will directly contact the bone surface as at least some cartilage may remain on the joint head if the implantation intervention is performed during early stages of joint damage or failure. Either or both of the exterior articulating region 41A and a bone contacting region 41B may themselves include more than one layer if desired.

Exterior articulating region 41A comprises a biocompatible polymer adapted to swell upon absorption of synovial fluid which swells at an initial rate of between 0.05 to 1.0 ml/g/min for a sheet section having a pre-saturated thickness of between 0.5 mm to 7.5 mm. The exterior articulating region 41A typically is substantially fully saturated with synovial fluid within 30 minutes of implantation and may be pre-saturated with sterile water or saline to facilitate implantation. During saturation, the exterior articulating region swells by 0.5-50% to fill in any dead spaces in the joint contact region. The swollen polymer of the exterior articulating region 41A is adapted to provide a low coefficient of friction and to resist wear when articulated against articular cartilage and/or subchondral bone of the acetabulum or the equivalent components of another joint such as a shoulder joint. During use, the exterior articulating region 41A sufficiently replenishes with synovial fluid between loading cycles experienced during normal physical activity such as walking or running. The fluid displaces under compressive loads and replenishes at a rate of 0.10-1.0 ml/g/min between loading cycles.

The polyether-urethane (PEU) and polyether-urethane-urea (PEUU) elastomer materials of the disclosure swell when placed in the biological environment by between about 1.5% to about 250% in volume. In some embodiments the polymer undergoes at least about 30% increase in volume by virtue of having a highly hydrophilic polyalkylene oxide as an inherent part of their segmented chain molecules. In particular, a hydroswellable, segmented, aliphatic polyurethane-urea comprising polyoxyalkylene chains covalently interlinked with polyalkylene urethane chain segments, which are further interlinked with aliphatic urea chain segments, may exhibit at least 50% increase in volume when placed in the biological environment. The PEUU materials tested were found to have 60% to 91% increase in volume after immersion in 1% methyl cellulose solution (to simulate synovial fluid viscosity) for 15 hours at 37° C.

Polyalkylene glycol chains can comprise at least one type of oxyalkylene sequences selected from the group represented by oxyethylene, oxypropylene, oxytrimethylene, and oxytetramethylene repeat units and the urethane chain segments are derived from at least one diisocyanate selected from the group represented by hexamethylene diisocyanate, heptamethylene diisocyanate, octamethylene diisocyanate, decamethylene diisocyanate, dodecamethylene diisocyanate, 1,4 cyclohexane diisocyanate, lysine-derived diisocyanate, and cyclohexane bis(methylene isocyanate). The resulting polyoxyalkylene urethane molecules, which may have at least one isocyanate terminal group, are chain-extended with an alkylene diamine selected from the group represented by ethylene-, trimethylene, tetramethylene-, hexamethylene-, and octamethylene-diamine, thus forming polyether urethane-urea segmented chains.

The useful polycarbonate urethanes (PCU), segmented polyether urethanes, polyether-urethane (PEU), polyether-urethane-urea (PEUU) elastomer materials or hydrogels including polyvinyl alcohol, polyacrylic acid, or polyethylene glycol for artificial cartilage polymer caps may be synthesized from, for example, the materials indicated above or purchased commercially. Representative commercial materials include Bionate® and Bionate® II thermoplastic polycarbonate urethanes, Biospan® segmented polyether urethane, and Elasthane™ polyether urethane (all from DSM Biomedical, Berkeley, Calif.); Chronoflex, Chronothane, Hydrothane, and Hydromed (all from AdvanSource Biomaterials, Wilmington, Mass.); and Carbothane, Isoplast, Tecoflex, Tecophilic and Tecothane (all from The Lubrizol Corporation, Wickliffe, Ohio).

A membrane cap corresponding to the peripheral geometry of a joint can be formed by known techniques such as dip molding followed by removal of any solvent and/or thermosetting the material on a mold form. In other embodiments, injection molding, including reactive injection molding, may be used to form the polymer cap. The membrane polymer cap can then be installed on the joint in a surgical procedure as described herein.

In some embodiments, the size and shape of the femoral head may be determined, for example by conventional three-dimensional imaging techniques, and a customized mold may be produced. Depending upon the molding process to be used, the mold may larger, smaller, or the same size as the femoral head to be covered. In certain embodiments, the size and shape of the mold and resulting polymer cap may be adjusted to accommodate residual cartilage associated with the femoral head and/or the acetabulum or corresponding components of other joints.

Biocompatible polymers adapted for use as the bone contacting region 41B typically may be firmer than the hydrated polymer of the exterior articulating region, although they also may be selected to swell upon hydration by synovial fluid if desired. Typically the bone contacting region may have a Shore hardness in the range of 40 A to 70 D and a 50% secant modulus of between 0.7 and 1.3 MPa. The bone contacting region 41B may range from about 0.50 mm to about 7.50 mm in thickness and the thickness may vary within the bone contacting region. The bone contacting region 41B is compliant enough to provide a degree of shock absorption and reduce patient pain while remaining stiff enough to resist implant malfunction due to buckling, deformation, or displacement under sheer stress. In addition, the bone contacting region 41B may resist implant rupture and allows the implant to remain intact as the articulating region wears thin and possibly tears. By remaining intact after the articulating region wears thin or tears, the patient is able to detect that the joint augmentation implant is nearing the end of its effective life without a more traumatic malfunction and bone on bone contact.

Suitable polymers for the bone contacting region 41B may include polymers suitable for exterior articulating region 41A or may be different. In addition to the materials described above, silicone containing polycarbonate urethanes, polyether ether ketone, polyvinyl pyrolidone, polyethylene or other biocompatible polymers may be used. Representative commercial materials include Carbosil® thermoplastic silicone polycarbonate urethanes and PurSil™ silicone polyether urethane (both from DSM Biomedical, Berkeley, Calif.), and Chronoprene and Chronosil (both from AdvanSource Biomaterials, Wilmington, Mass.).

The exterior articulating region 41A and bone contacting portion 41B may be joined by conventional means such as injection molding, solvent casting, grafting, and the like. In some embodiments, an optional interfacial layer 41C (FIG. 12A) may be used to facilitate bonding between the exterior articulating region and the 41A and bone contacting portion 41B. In other embodiments, the interface between exterior articulating region 41A and bone contacting portion 41B may include biocompatible reinforcing fibers, knits, braids, and the like. These reinforcing materials 60 may be directly interposed between the exterior articulating region 41A and bone contacting portion 41B or may be included, at least in part, in an interfacial layer 41C, shown in FIG. 12B. It will be appreciated that such reinforcements 60, if present, will typically be present around the entire circumference of the joint augmentation implant 40 and may extend across the spherical cap 41 and/or may extend into the neck 43 if desired. In other embodiments, the optional reinforcing fibers 60 may be confined to one or more of the component layers or portions thereof.

Reinforcing fibers 60 may allow the polymer cap 40 to stretch over the joint head without exceeding the elastic limit of the composite polymer cap 40 or allowing it to tear. Suitable biocompatible materials for the reinforcing fibers 60 include polyethylene terephthalate, polyamide, polyether ether ketone, polypropylene, carbon fibers, and combinations thereof. The fibers may be present as individual fibers, nonwovens, meshes, knit fabrics, or braids. As noted earlier, the fibers may be present in any or all of the three regions 41, 42, and or 43, of polymer cap 40. When reinforcing fibers 60 are present, not all regions of the polymer cap 40 need have the same reinforcing fiber(s) 60. Reinforcing fibers 60 may be incorporated by casting, insert molding, dipping or the like. Reinforcing fibers 60 may optionally be present when the polymer cap 40 is otherwise formed from a single optimized polymer. In some embodiments, the reinforcing fibers may be present as one or more circumferential bands, typically at the largest diameter of the polymer cap 40 or within the neck region 43. In certain embodiments, the fibers 60 may be distributed within bone contacting portion 41B rather than at the interface between exterior articulating region 41A and bone contacting portion 41B. In such embodiments the presence of the fibers 60 may serve to differentiate the compositions of exterior articulating region 41A and bone contacting portion 41B.

Neck region 43 forms a generally cylindrical cuff which surrounds the neck of the ball portion of the ball and socket joint when the joint augmentation implant 40 is in place. As noted previously, the neck region 43 may include a slight flare or chamfer (difficult to see in the figures) to assist in the step of spreading the neck portion 43 as the implant is installed over the ball of the joint. Suitable biocompatible materials for the neck region 43 include thermoplastic urethanes such as polycarbonate urethanes, silicone containing polycarbonate urethanes segmented polyether urethanes, polyether urethanes, polyether urethane ureas, or silicone rubbers. The neck region 43 has an elongation to break of greater than 400% and a flexural stress at 5% deflection of between 0.13 and 1.38 MPa when saturated. Although it is not necessarily the case that neck region 43 is under tension when the intra-articular joint augmentation implant 40 is positioned around the joint head, a mild degree of tension is typically present and desirable. To this end, the inner diameter of the neck region 43 may vary from substantially equal to the diameter of the bone neck to 50% undersized.

In some embodiments, the generally cylindrical neck region 43 may be formed separately and may include a portion of reinforcing fibers 60 which may then extend into intermediate generally spherical segment 42 and even into spherical cap 41.

In use, the intra-articular joint augmentation implant is typically pre-hydrated with sterile water or saline to initially swell the polymer(s) adapted to swell upon absorption of synovial fluid thereby increasing flexibility and reducing the force necessary to stretch the hollow polymer cap 40. In some procedures, the hollow polymer cap 40 may be stored in water or saline prior to implantation. In certain procedures, more than one polymer cap 40 may be available and hydrated to allow the surgeon to select a polymer cap 40 from a set of related polymer caps based upon the size and condition of the ball and socket of the joint to be repaired. For example, polymer caps 40 having a range of cap thicknesses may be available as a kit from which the surgeon may select a polymer cap 40 having a thickness which better matches the desired restored joint space. During implantation, the head of the ball of a ball and socket joint is exposed surgically through an access wound and by dislocation and/or detaching one side of the joint capsule. The hollow polymer cap is urged over the ball of the dislocated ball and socket joint which initially expands the neck region 43 of the polymer cap so that the neck region 43 may pass over the maximum diameter of the ball, for example the femoral head or humeral head, without plastic yielding and recover elastically as the neck region 43 contracts around the neck of the ball of the joint. The capped head of the ball of the ball and socket joint is then repositioned within the socket with the polymer cap 40 positioned between the ball and socket, whereupon the joint capsule is restored and the access wound is surgically closed.

Depending upon the condition of the joint when it is exposed, native cartilage may be present on one or both of the ball and socket of the joint to be restored. Typically, neither the surface of the ball portion nor the surface of the socket portion of the joint are modified during the installation of polymer cap 40, which significantly decreases the total surgical procedure time, reduces trauma to surrounding tissue, and retains any healthy cartilage which remains within the joint. Accordingly, the ball and socket components of the joint may be substantially free of cartilage, may be partially covered by cartilage, or may be completely covered by native cartilage at the completion of the intra-articular joint augmentation implant procedure.

### Example 1

A polyether urethane urea (obtained from Poly-Med, Inc., Anderson, S.C.) was solvent cast from a fluoroalcohol to form a disk (6 mm by 30 mm diameter) which was evaluated using an OrthoPOD (AMTI, Watertown, Mass.) pin-on-disk testing system. Testing was carried out with the sample submerged in bovine serum. The polymer system had a coefficient of friction of 0.006 under a 2 MPa load. After 500,000 cycles, there was no macroscopic wear and the wear rate was approximately 20 mg per million cycles.

### Example 2

A polyether urethane urea (Poly-Med, Inc., Anderson, S.C.) was solvent cast from fluoroalcohol to form a polymer cap. The polymer cap was sterilized and subjected to cytotoxicity testing (MEM Elution using L-929 Mouse Fibroblast Cells with a HDPE control). The test cultures had a score of zero; discrete intracytoplasmic granules were observed; there was no cell lysis and no cell growth inhibition was observed.

### Example 3

A polyether urethane urea (Poly-Med, Inc., Anderson, S.C.) was solvent cast from fluoroalcohol to form a symmetric cap, having a wall thickness in the load bearing region of approximately 2 mm and a wall thickness of approximately 1.5 mm in the neck region, which was positioned over an anatomically correct femoral head. The cap was saturated in bovine serum to mimic natural synovial fluid and placed on a hard plastic femoral head which was then articulated against an AMTI Hip Simulator (AMTI, Watertown, Mass.) against an acrylic acetabulum with anatomical alignment. During testing, lubrication was maintained with 20 g sterile bovine serum protein per liter maintained at 37° C. Testing was based on ISO 14242-1 with a vertical peak load of 1000 N and a walking frequency of 1.0 Hz. Testing was interrupted every 500,000 cycles. No visible deformation was observed after 3 million cycles.

Although the illustrative examples described above relate to hip and shoulder prostheses, a joint augmentation implant as artificial cartilage and the method of installing a joint augmentation implant is also suitable more generally for restoring the function of other types of diseased or defective articulating joints in humans. For example, a joint augmentation implant may be adapted for use for restoring a knee joint. A joint augmentation implant may also be adapted for use for restoring joints in mammals.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A joint augmentation implant for mammalian ball and socket joints comprising:
a hollow polymeric cap formed at least in part from a polymer adapted to swell upon absorption of synovial fluid;
wherein the hollow polymeric cap comprises a distal region having the general form of a spherical cap, a generally cylindrical proximal neck region, and an intermediate region comprising a generally spherical segment therebetween;
further wherein at least a portion of the hollow polymeric cap within the combined regions having the general form of a spherical cap and the intermediate region having the general form of a spherical segment comprises an exterior articulating region and a bone contacting region.

2. The joint augmentation implant of claim 1, wherein the polymer adapted to swell upon absorption of synovial fluid swells at an initial rate of between 0.05 to 1.0 ml/g/min for a sheet section having an unswollen thickness of between 0.5 mm to 7.5 mm.

3. The joint augmentation implant of claim 1, wherein the exterior articulating region and the bone contacting region comprise the same polymer.

5. The joint augmentation implant of claim 1, wherein the exterior articulating region and the bone contacting region comprise different polymers.

6. The joint augmentation implant of claim 1, wherein the exterior articulating region and the bone contacting region are at least partially separated by a material comprising a plurality of fibers, a knit, or a braid.

7. The joint augmentation implant of claim 1, wherein the generally cylindrical proximal neck region is sufficiently elastic to stretch over the ball of a ball and socket joint and to elastically recover when positioned about a neck associated with the ball without undergoing plastic yielding.

8. The joint augmentation implant of claim 1, wherein the water content of the polymer adapted to swell upon absorption of synovial fluid is between 0.5% and 80% of the weight of the polymer when saturated by water.

9. The joint augmentation implant of claim 1, wherein the bone contacting region has a Shore hardness between 40 A and 70 D.

10. The joint augmentation implant of claim 1, wherein the bone contacting region has a 50% secant modulus of between 0.7 and 1.3 MPa.

11. The joint augmentation implant of claim 1, wherein the proximal neck region has an elongation to break of at least 400% when saturated.

12. The joint augmentation implant of claim 1, wherein the proximal neck region has a flexural stress at 5% deflection of between 0.13 and 1.38 MPa when saturated.

13. A method of installing an intra-articular joint augmentation implant as artificial cartilage in a ball and socket joint to be restored comprising:
forming a hollow polymeric cap formed at least in part from a polymer adapted to swell upon absorption of synovial fluid, said hollow polymeric cap comprising a distal region having the general form of a spherical cap, a generally cylindrical proximal neck region, and an intermediate region comprising a generally spherical segment therebetween,
wherein the hollow polymeric cap within the combined regions having the general form of a spherical cap and the intermediate region comprises an exterior articulating region and a bone contacting region;
hydrating the hollow polymeric cap;
surgically exposing the head of the ball of the ball and socket joint through an access wound;
installing the hollow polymeric cap over the head of the ball of the ball and socket joint;
repositioning the capped head of the ball of the ball and socket joint within the socket of the ball and socket joint with the hollow polymeric cap interposed between the ball of the ball and socket joint and the socket of the ball and socket joint; and
surgically closing the access wound.

14. The method of claim 13, wherein the polymer adapted to swell upon absorption of synovial fluid loses absorbed water upon compressive mechanical loading and regains water at a rate of between 0.10 to 1.00 ml/g/min upon becoming unloaded.

15. The method of claim 13, wherein the bone contacting region has a 50% secant modulus of between 0.7 and 1.3 MPa.

16. The method of claim 13, wherein the generally cylindrical proximal neck region is sufficiently elastic to stretch over the ball of a ball and socket joint and to elastically recover when positioned about a neck associated with the ball without undergoing plastic yielding.

17. The method of claim 13, wherein the ball of the ball and socket joint is unmodified prior to the installing step.

18. The method of claim 13, wherein the socket of the ball and socket joint is unmodified prior to the installing step.

19. The method of claim 18, wherein the ball and socket joint is a mammalian socket joint.
